(19) United States
(12) Patent Application Publication
(10) Pub. No.: US 2025/0275776 A1
(43) Pub. Date: Sep. 4, 2025

(54) Drill Guides For Bone Plates

(71) Applicant: Stryker European Operations Limited, Carrigtwohill (IE)

(72) Inventors: Lance N. Terrill, Rochestown (IE); Jonathan David Forde, Renmore (IE); Matthias Paulisch, Roggwil (CH); Ana Trapero Martin, Cambridge, MA (US); Jerome Mutschler, Neuchâtel (CH)

(21) Appl. No.: 19/065,262

(22) Filed: Feb. 27, 2025

Related U.S. Application Data
(60) Provisional application No. 63/560,240, filed on Mar. 1, 2024.

(51) Int. Cl. A61B 17/17; A61B 17/16

(52) U.S. Cl. CPC A61B 17/1728; A61B 17/1633; A61B 17/171

(57) ABSTRACT

The present disclosure is directed to drill guides that provide for better cooperation with plate holes. The drill guides according to the present disclosure cooperate with plate holes in a manner that prevents both rotational and translational movement between the components. In certain aspects, the guides can be designed to permit a drill to be placed over a range of angles, while in other aspects, the drill angle can be fixed.

### CROSS-REFERENCE TO RELATED APPLICATION

This application claims priority to the filing date of U.S. Provisional Patent Application No. 63/560,240, filed Mar. 1, 2024, the disclosure of which is hereby incorporated herein by reference.

### BACKGROUND

The present disclosure relates to drill guides for use in surgical procedures. While discussed largely in connection with use in bone plates, the present disclosure is also applicable to other types of medical implants.

Bone plates are conventionally secured to a bone using a plurality of screws placed through holes in the bone plate and into the bone. Prior to the insertion of the screw, and to prevent unwanted damage to the underlying bone, a drilling process is often performed. While it is possible to freehand drill, many surgeons prefer the precision offered by the use of a drill guide that dictates or otherwise limits the positioning of a drill.

For instance, one type of conventional drill guide includes a variable angle cone, which is designed to permit varying drill angles while preventing the angle of the drill from exceeding a specified point, depending on the radius of the cone. Prior art variable angle drill guides often suffer from a poor cooperation with the bone plate that permits the guide to rock within the plate hole. This, sometimes significantly, impacts the outer limits of drill angulation. Accordingly, surgeons often refer to variable angle cones as “cones of uncertainty.”

Another conventional drill guide is a fixed angle guide, which is designed to restrict the angle of the drill to a particular angle, e.g., 0°. Prior art fixed angle guides often fall into two buckets—designs that thread into the plate hole and designs without threading mechanisms. The former are often difficult to use because the threads are easily cross threaded, while the latter are often not stable and therefore do not accurately limit the angle of the drill to the desired angle.

Accordingly, there is a need for drill guides with improved angular precision.

### BRIEF SUMMARY

The present disclosure is directed to different drill guides with better cooperation with plates. For instance, the drill guides disclosed herein include anti-rotational features for cooperation with a plate hole, as well as other more positive connections between the guides plates. This results in more assurances that the guides are being utilized correctly in the course of a surgical procedure.

In one aspect of the disclosure, a drill guide has a first end, a second end, and a passage extending from the first end to the second end. The second end is compressible and includes anti-rotational features for cooperation with a plate hole.

In some examples, the second end includes at least one cutout. The at least one cutout is a plurality of cutouts. The at least one cutout extends from the second end towards the first end at an angle in relation to a longitudinal axis of the drill guide. In other examples, the at least one cutout includes a reverse angle to helix angle.

In some examples, the second end compresses when the second end is partially inserted into a plate hole of a bone plate and expands when the second end is fully inserted into the plate hole.

In some examples, the anti-rotational features on the second end include a plurality of protrusions. The plurality of protrusions are eight or more protrusions. The plurality of protrusions abut structures in a hole of a bone plate.

In some examples, the second end is frustoconical. In other examples, the second end is cylindrical.

In some examples, the second end includes a flexible material capable of compression upon insertion into the plate hole.

In another aspect of the disclosure, a system for angling a drill has a bone plating system and a drill guide including a first end, a second end, and a passage extending from the first end to the second end. The bone plating system has a plate having a bone-contacting surface and an upper surface, a plate hole through the bone-contacting surface and the upper surface, a plurality of scalloped regions on a surface of the plate hole, and a plurality of screw-engaging members on an inner surface of the plate hole. The second end is compressible and includes anti-rotational features for cooperation with the plurality of screw-engaging members when the second end is received within the plate hole. In some examples, the anti-rotational features of the drill guide include a plurality of protrusions abutting the plurality of screw-engaging member when the second end is received within the plate hole. The anti-rotational features of the drill guide include resisting forces between the second end of the drill guide and the inner surface of the plate hole, wherein the resisting forces are axial forces towards and from a midpoint of the drill guide.

In some examples, a drill is inserted through the passage of the drill guide and through the plate hole. In some examples, the drill is inserted through the passage at an angle ranging from approximately 0° to 15° from an axis perpendicular to a center of the plate hole. In other examples, the drill is inserted through the passage along an axis perpendicular to a center of the plated hole.

In some examples, the drill guide includes a drill insert configured to be received in a drill sleeve.

In some examples, an outer surface of the drill guide includes a knurl pattern having a groove width ranging from approximately 0.9 mm to 1.5 mm.

In another aspect of the disclosure, a method for angling a drill includes inserting a drill guide in a plate hole of a bone plate. During insertion, a portion of the drill guide compresses and cooperates with the plate hole to prevent translational and rotational movement of the drill guide with respect to the bone plate. The method also includes inserting a drill through a passage in the drill guide and into a bone underlying the bone plate.

In some examples, the method also includes the step of angulating the drill with respect to the plate hole.

In some examples, the angulation ranges from approximately 0° to 15° from an axis perpendicular to a center of the plate hole.

### BRIEF DESCRIPTION OF THE DRAWINGS

FIG. 1A is a perspective view of a drill guide in accordance with an aspect of the present disclosure.

FIG. 1B is an enlarged view of a portion of the drill guide of FIG. 1A.

FIGS. 2A and 2B are right and left side views of the drill guide of FIG. 1A.

FIGS. 3A and 3B are right and left bottom perspective views of the drill guide of FIG. 1A.

FIGS. 4A and 4B are bottom and top plan views of the drill guide of FIG. 1A.

FIG. 5A is a perspective view of a bone plate for use with the drill guide of FIG. 1A.

FIG. 5B is a perspective view of the drill guide of FIG. 1A engaged with the bone plating system of FIG. 5A.

FIG. 6 is a cross-sectional view of the drill guide and bone plating system taken along axis X of FIG. 5B.

FIG. 7 is a top perspective view depicting a drill placed through the drill guide and plate construct of FIG. 5B.

FIG. 8 is a bottom perspective view of the system of FIG. 7.

FIG. 9 is a side view of a drill guide in accordance with another aspect of the present disclosure.

FIG. 10 is a side exploded view of the drill guide of FIG. 9.

FIGS. 11A and 11B are bottom and top plan views of the drill guide of FIG. 9.

FIG. 12 is a side perspective view of the drill guide of FIG. 9 engaged with the bone plate of FIG. 5A.

FIG. 13 is a cross-sectional view of the drill guide and bone plating system taken along axis X of FIG. 12.

FIG. 14 is a bottom plan view of the drill guide and bone plating system of FIG. 12.

FIG. 15 is a side view depicting a drill placed through the drill guide and plate construct of FIG. 12.

FIG. 16 is a cross-sectional view of the system taken along axis X of FIG. 15.

FIG. 17 is a side perspective view of an attachment method for the drill guide and plate construct of FIG. 12.

### DETAILED DESCRIPTION

Particular aspects of the present disclosure will be described herein with reference to the accompanying drawings. As shown in the drawings and as described throughout the following description, and as is traditional when referring to relative positioning on an object, the term “proximal” should be understood as referring to the portion of a structure that is closer to a clinician during proper use and the term “distal” should be understood as referring to the portion of a structure that is farther from the clinician during proper use. Also, as used herein, the terms “substantially,” “generally,” and “about” are intended to mean that slight deviations from absolute are included within the scope of the term so modified.

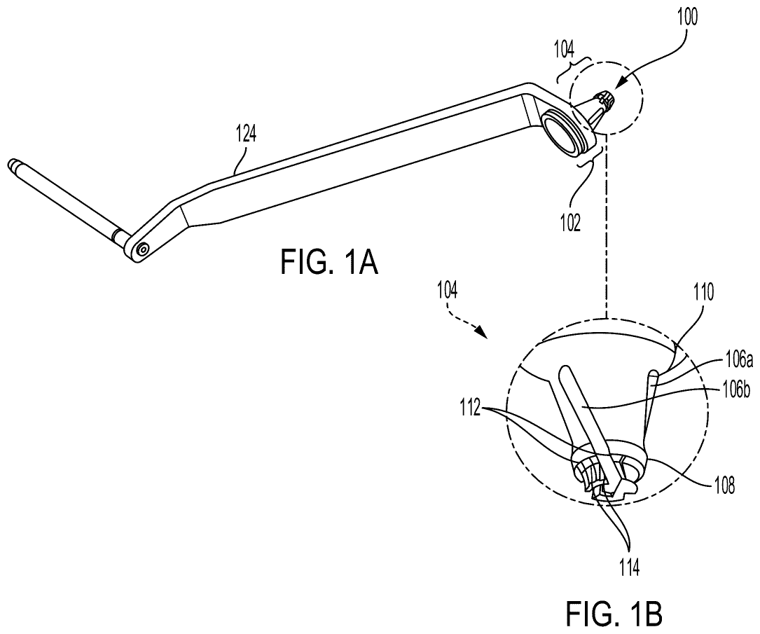

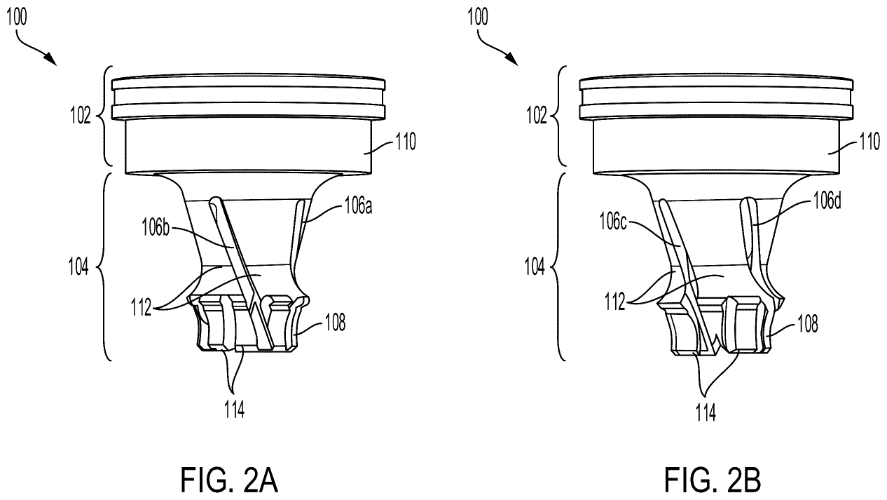

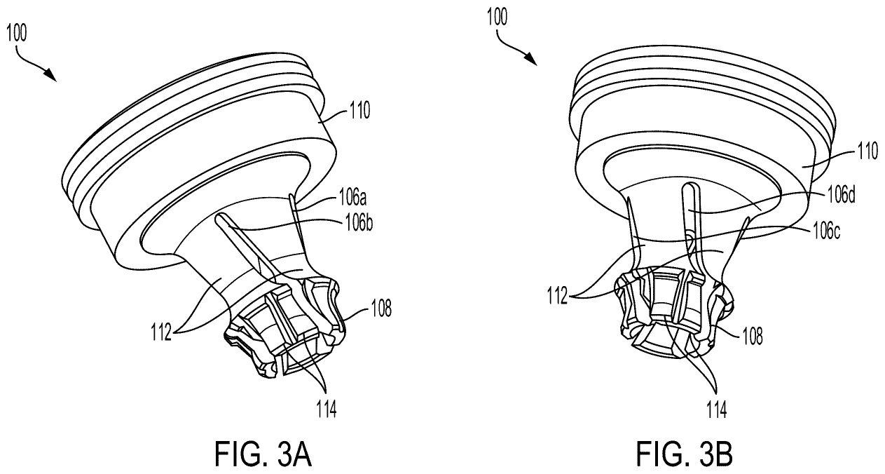

FIGS. 1A-4B depict a drill guide 100 in accordance with an aspect of the present disclosure. Drill guide 100 is a variable angle drill guide and permits a drill to be placed therethrough and through a plate hole at an angle ranging from approximately 0° to 15° from an axis perpendicular to a center of a plate hole on a bone plate. This is discussed more fully below. As shown, drill guide 100 includes a first end 102, a second end 104, cutouts 106a and 106b, a tip 108, a base 110, a plurality of sides 112, a plurality of protrusions 114, and a passage 122.

First end 102 is connected to second end 104 and a passage 122 extends through both ends. Passage 122 is configured to receive a drill at various angles, as further described below. First end 102 is substantially cylindrical, and second end 104 is frustoconical with the end with the smallest radius facing away from first end 102.

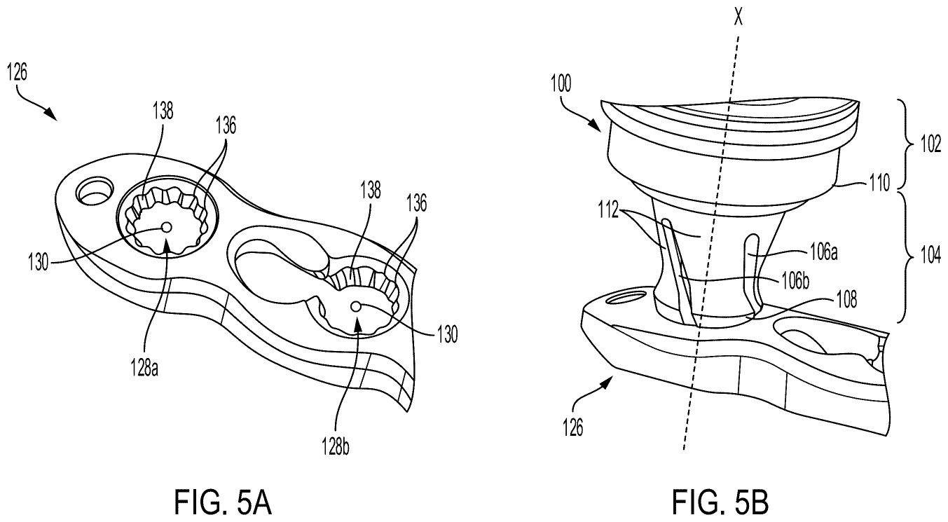

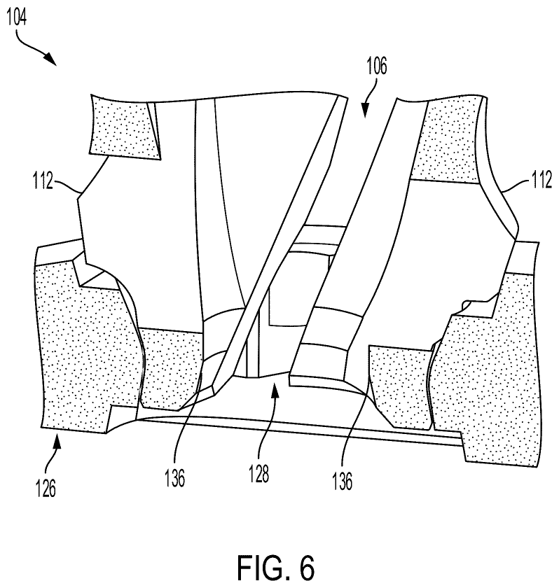

Cutouts 106a-d extend from tip 108 to base 110 of drill guide 100. Cutouts 106a-d include a reverse angle to helix angle to reduce the risk of inadvertent drill contact with tip 108, as this could potentially break or otherwise impact the structural integrity of the drill. In other words, the inclusion of the reverse helix design reduces the chance of a drill landing on an end of a conical drill guide. These cutouts permit at least a portion of second end 104 to be compressible/expandable. In particular, a plurality of sides 112 are located between cutouts 106 and compress upon insertion into a plate hole, as further described in connection with FIGS. 5B-6.

Though the aspect drill guide shown in the drawings includes four cutouts, a drill guide in accordance with the present disclosure may have any number of cutouts. There also may be no cutouts on a drill guide. For example, a drill guide may include a flexible material capable of compressing a portion of the drill guide upon insertion into a plate hole.

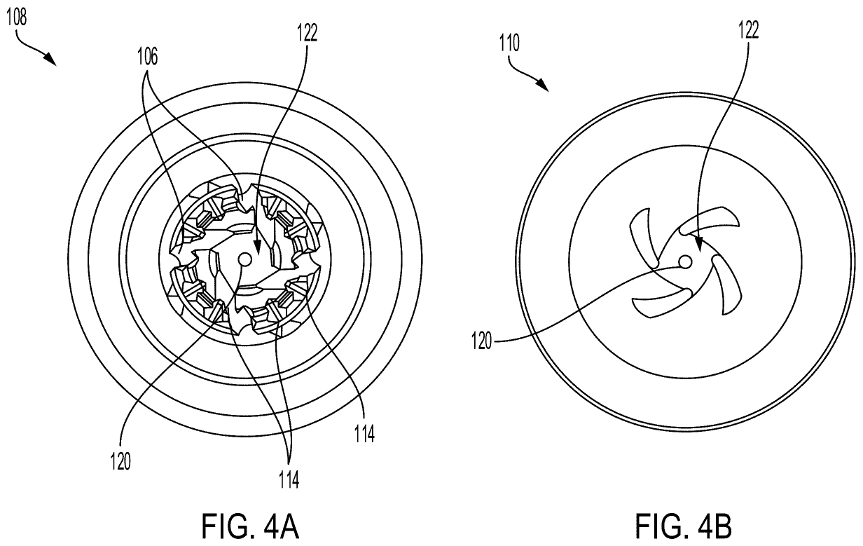

Drill guide 100 also includes a plurality of protrusions 114 extending along tip 108 and protruding away from a midpoint 120 of drill guide 100 (best shown in FIG. 4A). The protrusions 114 are located around the circumference of passage 122 on the bottom portion of drill guide 100. In the aspect shown, drill guide 100 includes twelve protrusions 114, four of which are intersected by cutouts 106. Protrusions 114 are configured to prevent drill guide 100 from moving translationally or rotationally in a plate hole, as further described in connection with FIGS. 5B and 6. Of course, in other aspects, drill guide 100 can include any number of protrusions suitable for cooperation with a given plate. Likewise, although shown as being of a particular shape for cooperating with corresponding structure of a plate (discussed below) the protrusions can exhibit any shape necessary to cooperate with a given plate.

Drill guide 100 is attached to handle 124, which is angled on each end to improve ergonomics. Handle 124 has a steep angle on the end closest to drill guide 100 to enable a drill to enter the small window due to the conical shape of drill guide 100. Additionally, the steep angle of handle 124 minimizes the moment arm and therefore limits the risk of drill guide 100 popping out of place after insertion in a plate hole. In the aspect shown, drill guide 100 is pressed into an opening in handle 124 and the two components are welded together. However, it is contemplated to utilize other connections between the two components, including removable connections such as snap fits or threading.

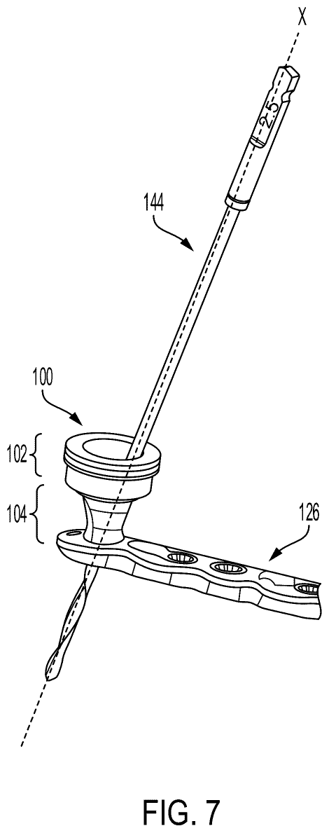

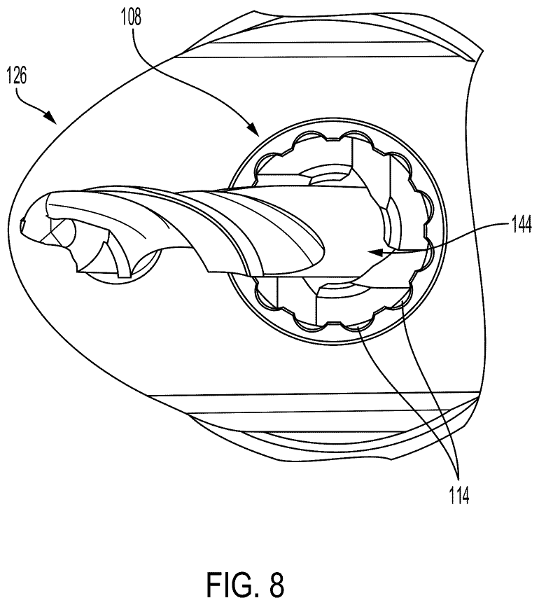

FIGS. 4A and 4B show bottom and top views of drill guide 100, respectively. Passage 122 is configured to receive a drill through the top portion of drill guide 100 in FIG. 4B to the bottom portion of drill guide 100 in FIG. 4A. This instrument can be inserted at an angle range from approximately 0° to 15° from an axis perpendicular to a center of a plate hole because of the fact that the passage is conical in nature. This is best show in, for instance FIGS. 7 and 8, which will be discussed more fully below.

FIG. 5A shows a bone-plating system 126 in accordance with an aspect of the present disclosure. Among certain common plate features, such as a bone-contacting surface (not shown), an opposing upper surface 111 and k-wire holes, bone-plating system 126 includes a plurality of plate holes, including locking holes 128a and locking-compression holes 128b. These holes are designed to receive locking screws at polyaxial orientations, and both include scalloped regions 136 along an inner surface 138. While these structures are generally referred to herein, the plate hole designs are more specifically disclosed in U.S. patent application Ser. No. 17/332,090 (“the '090 Application”), the disclosure of which is hereby incorporated by reference herein. It is also contemplated to employ any of the other plate hole designs disclosed in the '090 Application in accordance with the present disclosure.

FIGS. 5B and 6 show drill guide 100 engaged with bone-plating system 126 of FIG. 5A. Passage 122 is aligned substantially parallel to inner surface 138. When second end 104 is initially inserted into plate hole 128a, sides 112 between each cutout 106 compress towards midpoint 120 of drill guide 100. When second end 104 is fully inserted into plate hole 128a, plurality of sides 112 expand away from midpoint 120 of drill guide 100. This results in inner surface 138 and plurality of sides 112 exerting forces on each other thereby securing drill guide 100 in plate hole 128a both rotationally and translationally. In particular, protrusions 114 abut inner surface 138 of bone-plating system 126. A similarly cooperation would be seen between drill guide 100 and plate hole 128b.

FIGS. 7 and 8 show a drill 144 placed through drill guide 100 and bone-plating system 126. In the aspect shown, the conical shape of drill guide 100 limits drill 144 to an angle range from approximately 0° to 15° from an axis X perpendicular to a center 130 of plate hole 128a. In other words, drill guide 100 limits drill from extending past 15° angle in any direction away from midpoint 120 of drill guide 100. In other aspects, drill guide 100 can be configured to limit the angulation to any range that still permits a screw to be properly received in a given plate hole.

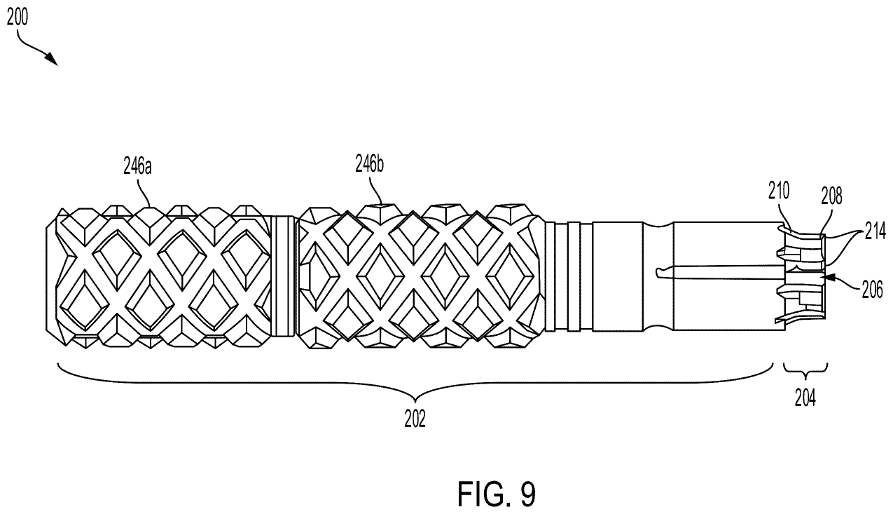

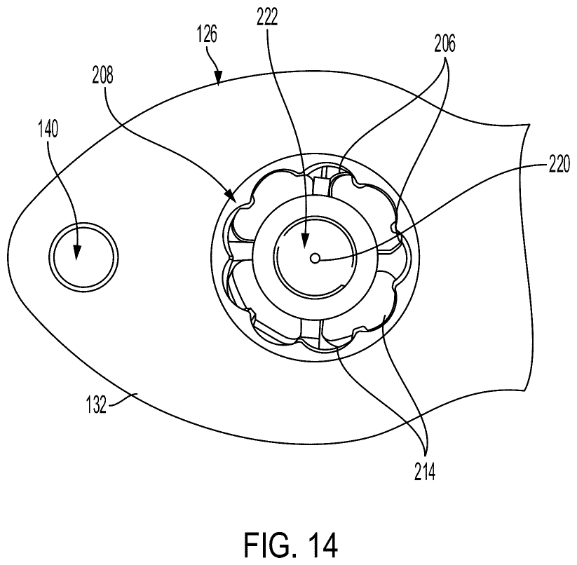

FIGS. 9-17 show a drill guide 200, in accordance with another aspect of the disclosure. In this aspect, drill guide 200 is similar to drill guide 100 of FIGS. 1A-8, and therefore like elements are referred to with similar numerals within the 200-series of numbers. Additionally, the description of certain similar features between drill guide 200 and drill guide 100 is omitted for brevity, with the following focusing on the differences between the two drill guides.

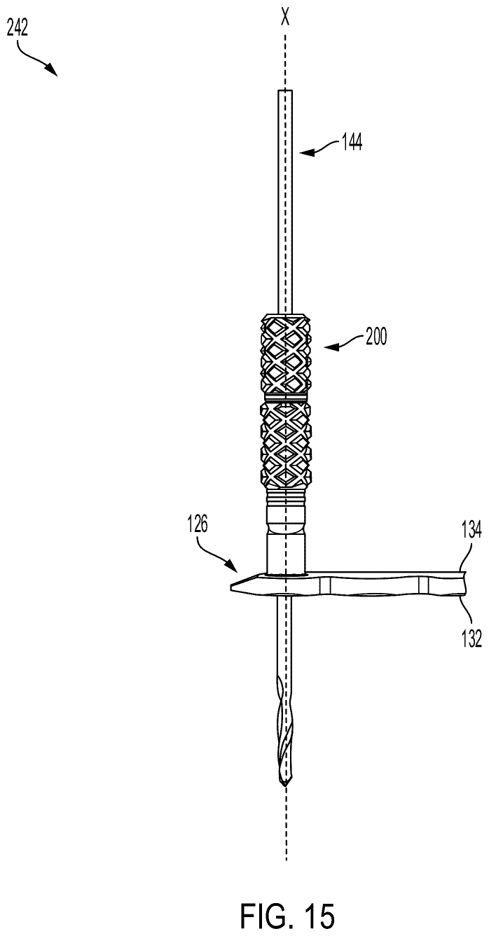

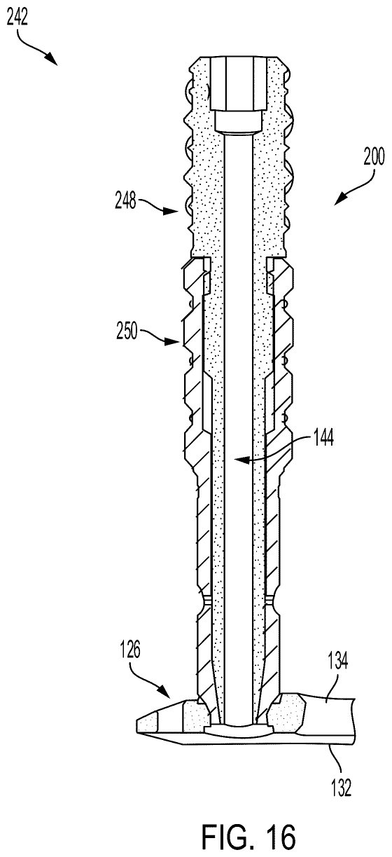

Drill guide 200 is used to position a drill at a fixed angle. As the aspect shows, the guide is design to only permit the drill to extend at a 0° angle perpendicular to a center of a plate hole, as best shown in FIGS. 15-16. In its fully constructed state, drill guide 200 includes a first end 202, a second end 204, a tip 208, a base 210, cutouts 206, a plurality of protrusions 214, and a passage 222. The cooperation of second end 204 of drill guide 200 is much like that of drill guide 100 except that a drill sleeve 250 is inserted into a plate hole and then a drill insert 248 is inserted through a passage in drill sleeve 250 to secure tip 208 in the plate hole. This mechanism ensures that a screw is accurately placed coaxially to a plate hole and ensures that drill guide 200 is secured enough to hold a bone plate for insertion. Likewise, passage 222 is designed to accept a drill much like in the first aspect, but again, at a fixed angle.

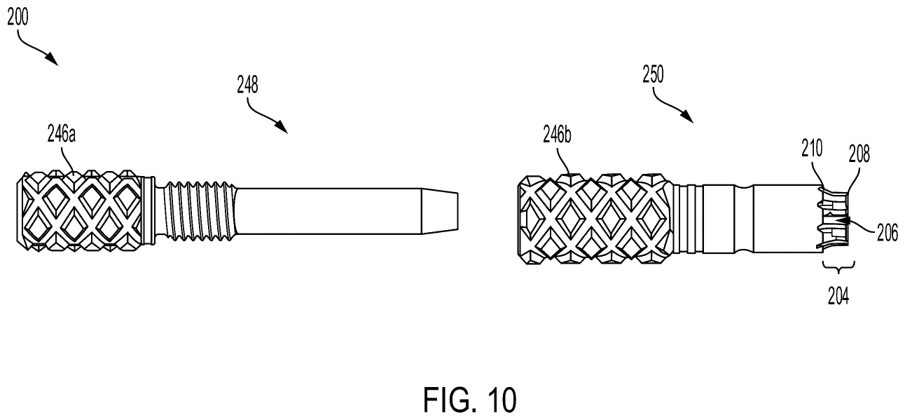

Knurl pattern 246a-b on drill guide 200 is designed to improve gripping performance by a surgeon or the like. The gaps between ridges are increased compared to conventional knurl patterns because knurl pattern 246a-b is created through a turning process that includes milling rather than with a knurling tool that includes grinding. The grinding process for conventional knurl patterns can result in contaminants being ground into the metal of a drill guide and can result in decreased gaps between ridges. Knurl pattern 246a-b has a groove width ranging from 0.9 mm to 1.5 mm. Knurl pattern 246a is located on drill insert 248, while knurl pattern 246a is located on drill sleeve 250, as best shown in FIG. 10. It has been found that this pattern has an increased gripping performance, even with gloved and/or wet hands. The particular groove width also provides for optimal gripping while reducing the potential for gloves or skin to catch on a ridge, thereby doing unwanted damage.

FIG. 10 is an exploded view of drill guide 200, which illustrates the drill insert 248 configured to be received in the drill sleeve 250. Drill insert 248 and drill sleeve 250 are connected through a threaded portion, i.e., screw-engaging members. In a fully connected configuration, a distal portion of drill insert 248 prevents any compression of second end 204 thereby locking the guide to the plate.

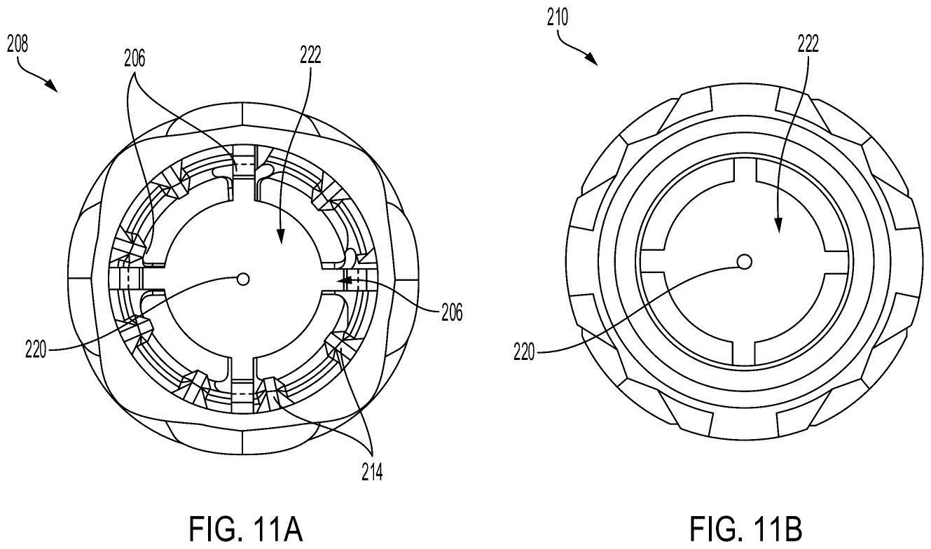

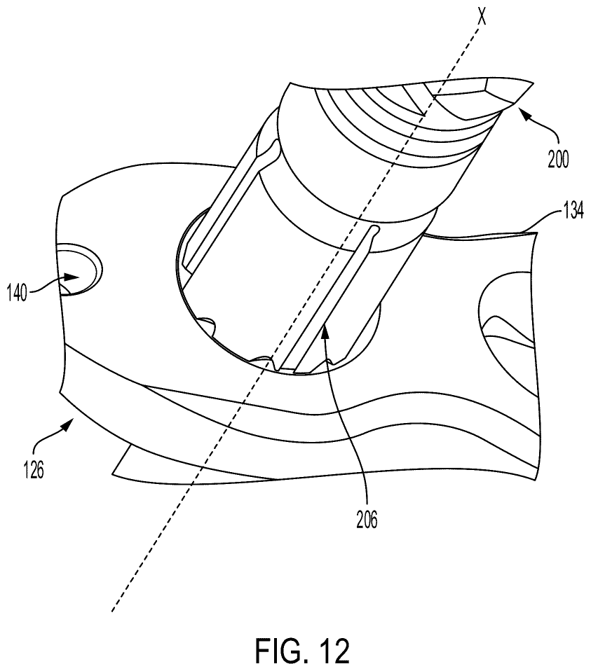

FIGS. 11A and 11B show top and bottom views of drill guide 200, respectively. Passage 222 is shown as being configured to receive a drill through the top portion of drill guide 200 in FIG. 12B to the bottom portion of the drill guide 200 in FIG. 12A, at a 0° angle perpendicular to a center of a plate hole, as further shown in FIGS. 15 and 16.

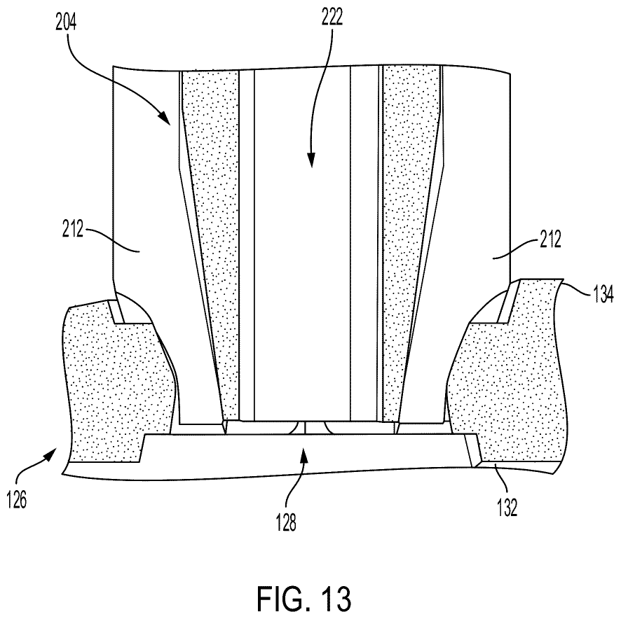

FIGS. 12-16 show drill guide 200 engaged with the bone-plating system 126 (shown in FIG. 5A). Again, the initial cooperation between the guide and plate hole is much like that is described above in connection with drill guide 100. However, upon insertion of drill insert 248, the guide is locked to the plate. In other words, drill sleeve 250 is first placed in cooperation with a plate hole and then drill insert 248 is placed to prevent any inadvertent removal of the guide from the plate. The cooperation between the insert and sleeve is shown in detail in the cross-sectional view of FIG. 13. As shown there, the insert effectively prevents any compression of the sleeve to allow for the removal of the guide from the plate.

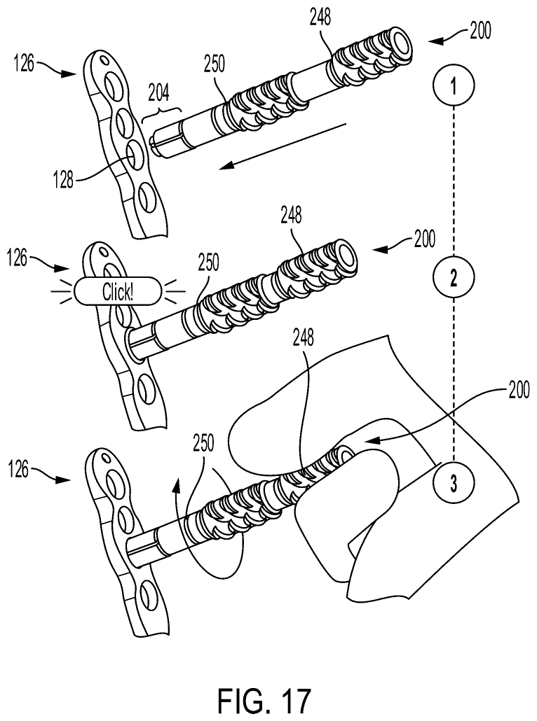

In a method of using either drill guide 100 or 200 according to the present disclosure, the particular guide is first engaged with a bone plate that has been placed on the bone. In the case of drill guide 200, this includes first engaging drill sleeve 250 and then placing drill insert 248 therein to lock the guide to the plate. Specifically, FIG. 17 shows a method of engaging drill guide 200 with bone-plating system 126. In step 1, second end 204 of drill sleeve 250 is inserted into plate hole 128 and is snapped into place. In step 2, drill insert 248 is further inserted into drill sleeve 250 to firmly lock drill guide 200 into plate hole 128. In step 3, drill insert 248 is rotated to threadably engage drill sleeve 250. The threaded portions of drill insert 248 and drill sleeve 250 are best shown in FIG. 10. In an alternative method, drill sleeve 250 could be inserted entirely separate from drill insert 248 and the two components could thereafter be attached together.

With drill guide 100 or 200 attached to the plate, a drill 144 is inserted through passage 122 or 222 from the first end 102 or 202 to the second end 104 or 204 and then through plate hole 128.

For drill guide 100, drill 144 can be angulated from approximately 0° to 15° from an axis X perpendicular to a center 130 of plate hole 128. Drill guide 100 may be attached to handle 124 by pressing drill guide 100 into an opening on handle 124, the case of aspects where the two components are not already fixed together. For drill guide 200 in FIGS. 9-17, drill 144 is fixed at a 0° angle from axis X. Drill 144 is then used to drill into bone for insertion of a screw. The guide is then removed from plate hole 128 by essentially performing the reverse of the insertion operation. At this point, a screw may be inserted into plate hole 128 using standard tools, such as a screwdriver. Again, this may occur according to the methodology disclosed in the '090 Application.

Drill guides according to the present disclosure may be constructed of any suitable material for their use in a surgical procedure like is described herein. For instance, drill guides 100 and 200 may be constructed of a metallic material, such as stainless steel or titanium. Preferably, the drill guides are also designed such that they can be sterilized and reused in multiple procedures. However, it is contemplated to construct a drill guide according to the present disclosure in a manner which permits it to be discarded after a single use. In such an instance, the drill guide could be constructed of a polymeric material or the like, although metals could also be employed in that instance. It is also contemplated to vary the materials utilized in the drill guides depending upon other structures thereof. For instance, the number of cutouts included in a guide may impact the material required to permit a portion of the guide to be compressible/expandable.

The disclosure set forth herein includes any possible combinations of the particular features set forth above, whether specifically disclosed herein or not. For example, where a particular feature is disclosed in the context of a particular aspect, arrangement, configuration, or arrangement, that feature can also be used, to the extent possible, in combination with and/or in the context of other particular aspects, arrangements, configurations, and arrangements of the technology, and in the technology generally.

Furthermore, although the technology herein has been described with reference to particular features, it is to be understood that these features are merely illustrative of the principles and applications of the present technology. It is therefore to be understood that numerous modifications, including changes in the sizes of the various features described herein, may be made to the illustrative arrangements and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. A drill guide comprising:
a first end;
a second end; and
a passage extending from the first end to the second end,
wherein the second end is compressible and includes anti-rotational features for cooperation with a plate hole.

2. The drill guide of claim 1, wherein the second end includes at least one cutout.

3. The drill guide of claim 2, wherein the at least one cutout is a plurality of cutouts.

4. The drill guide of claim 2, wherein the at least one cutout extends from the second end towards the first end at an angle in relation to a longitudinal axis of the drill guide.

5. The drill guide of claim 2, wherein the at least one cutout comprises a reverse angle to helix angle.

6. The drill guide of claim 1, wherein the second end compresses when the second end is partially inserted into a plate hole of a bone plate and expands when the second end is fully inserted into the plate hole.

7. The drill guide of claim 1, wherein the anti-rotational features include a plurality of protrusions.

8. The drill guide of claim 7, wherein the plurality of protrusions are eight or more protrusions.

9. The drill guide of claim 7, wherein the plurality of protrusions abut structures in a hole of a bone plate.

10. The drill guide of claim 1, wherein the second end is frustoconical.

11. The drill guide of claim 1, wherein the second end is cylindrical.

12. The drill guide of claim 1, wherein the second end comprises a flexible material capable of compression upon insertion into the plate hole.

13. A system for angling a drill comprising:
a bone plating system comprising:
a bone plate having a bone-contacting surface and an upper surface;
a plate hole through the bone-contacting surface and the upper surface;
a plurality of scalloped regions on a surface of the plate hole; and
a plurality of screw-engaging members on an inner surface of the plate hole; and
a drill guide comprising a first end, a second end, and a passage extending from the first end to the second end, wherein the second end is compressible and includes anti-rotational features for cooperation with the plurality of the screw-engaging members when the second end is received within the plate hole.

14. The system of claim 13, wherein the anti-rotational features of the drill guide include a plurality of protrusions abutting the plurality of the screw-engaging member when the second end is received within the plate hole.

15. The system of claim 13, wherein the anti-rotational features of the drill guide include resisting forces between the second end of the drill guide and the inner surface of the plate hole, wherein the resisting forces are axial forces towards and from a midpoint of the drill guide.

16. The system of claim 13, further comprising a drill inserted through the passage of the drill guide and through the plate hole.

17. The system of claim 16, wherein the drill inserted through the passage at an angle ranging from approximately 0° to 15° from an axis perpendicular to a center of the plate hole.

18. The system of claim 16, wherein the drill is inserted through the passage along an axis perpendicular to a center of the plated hole.

19. The system of claim 13, wherein the drill guide comprises a drill insert configured to be received in a drill sleeve.

20. The system of claim 13, wherein an outer surface of the drill guide comprises a knurl pattern having a groove width ranging from approximately 0.9 mm to 1.5 mm.
